(19) United States
(12) Patent Application Publication
(10) Pub. No.: US 2020/0179154 A1
(43) Pub. Date: Jun. 11, 2020

(54) HEAD SUPPORT

(71) Applicants: James Stafford, Gilroy, CA (US); Roger Minkow, Petaluma, CA (US); Robert Egger, Watsonville, CA (US); Piet Van Der Velde, Ommen (NL)

(72) Inventors: James Stafford, Gilroy, CA (US); Roger Minkow, Petaluma, CA (US); Robert Egger, Watsonville, CA (US); Piet Van Der Velde, Ommen (NL)

(21) Appl. No.: 16/703,615

(22) Filed: Dec. 4, 2019

Related U.S. Application Data
(60) Provisional application No. 62/775,743, filed on Dec. 5, 2018.

(51) Int. Cl. A61F 5/055; A47C 16/00; A47C 7/38

(52) U.S. Cl. CPC A61F 5/055; A47C 7/383; A47C 16/00

(57) ABSTRACT

A head support includes a chin member, a chest member, a multi-position connection, and a strap. The chin member includes a chin engagement surface. The chest member includes a chest engagement surface. The multi-position connection is located between the chest member and the chin member, and is adapted to maintain an angle between the chest member and the chin member in a collapsed angle of a collapsed configuration and in a plurality of expanded angles greater than the collapsed angle. The multi-position connection includes an angle adjustment actuator adapted to permit the angle between the chest member and the chin member to be changed or maintained. The strap is configured to extend from first and second sides of the chest support and around the user's neck or head when the chest member is engaged with the user's chest.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Air travel is long and uncomfortable. The flights are generally full, so each passenger has only his or her seat. In the economy sections the seats are upright or recline at most around 20 degrees. If the passenger attempts to sleep, his or her head tends to drop forward or to the side which can lead to discomfort preventing sleep or creating pain in the neck or arms. Travelers with spinal disease in their cervical spines are even more likely to have these problems.

In order to ease the neck discomfort, travelers have purchased neck cushions that are padded in the back of the neck and on the sides. These cushions do not hold the head up and do not provide enough padding on the sides to support the head in an upright position. At the other extreme are prior neck supports that provide a completely rigid support with no give at all.

Soft cervical collars have been used to relieve discomfort but offer only partial support in holding up the head.

Hard cervical supports, as found in the medical supply houses, are meant to brace the head and neck and keep the head from moving at all. These supports are large, bulky and uncomfortable. The position of the head using one of these clam-shell hard supports is approximately 90 degrees which is not practical when the chair is reclined.

### SUMMARY OF THE DISCLOSURE

The reason for cervical discomfort while attempting to sleep in an upright position (e.g., while traveling upright in an airline seat) is shown in FIG. 1. The human head weighs approximately 14 lbs. The center of gravity of the skull is shown at A, while B shows the center of the cervical spine. As shown at C, there is approximately 0.25 ft between these two points so that there is approximately 3.51b-ft of torque forcing the head to fall forward. This is normally countered by the posterior cervical musculature (trapezius, splenius, longissimus capitis, levator scapulae, semispinalis capitis, and other smaller muscles) to hold the head in an upright position while the person is awake. If the user sleeps, the muscles relax and the head falls into a flexion position that opens the disc spaces in the posterior aspect of the cervical spine and stretches the posterior ligaments of the spine. For people who have musculo-skeletal cervical disease, sleeping with the head forward and the cervical spine in flexion can worsen their symptoms by increasing intra-discal pressure in the cervical discs and stretching the posterior ligaments of the cervical spine. Cushions or pillows that wrap around the back and sides of the neck have been used to allow users to more comfortably rest and sleep, but are effective only when the user is extremely reclined. These cushions offer no support when the user is upright.

The head support of this invention is light weight while offering solid support to support the head. In one embodiment, the head support has a chin member and a chest member connected via a multi-position connection that maintains the angle between the chest member and the chin member in a collapsed angle and a plurality of expanded angles greater than the collapsed angle. Using this device, the traveler can sleep without fear of the head falling into an uncomfortable position. The head support of this invention counters the forces that tend to rotate the head forward by supporting the user's chin and holding the head up from the front.

The head support of this invention is the first cervical support that is lightweight, compact, non-medical looking which offers enough rigidity to support the weight of the head, allowing relaxation of the posterior cervical musculature. This device allows the traveler to rest or sleep while in an upright, seated position.

For spinal patients, the head support of this invention can be used when absolute head stabilization is no longer necessary but some support is still needed during rehabilitation. The patient who wants to sit in a recliner and watch television can use this support and be much more comfortable.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows the orthopedic anatomy of the neck and head.

FIG. 2 is a perspective view of a head support according to an embodiment of the invention in an expanded configuration.

FIG. 3 is a side view of the head support of FIG. 2 in an expanded configuration.

FIG. 4 is a perspective view of the head support of FIG. 2 in a collapsed configuration.

FIG. 5 is an exploded view of the head support of FIG. 2.

FIG. 6 shows the head support of FIG. 2 in use.

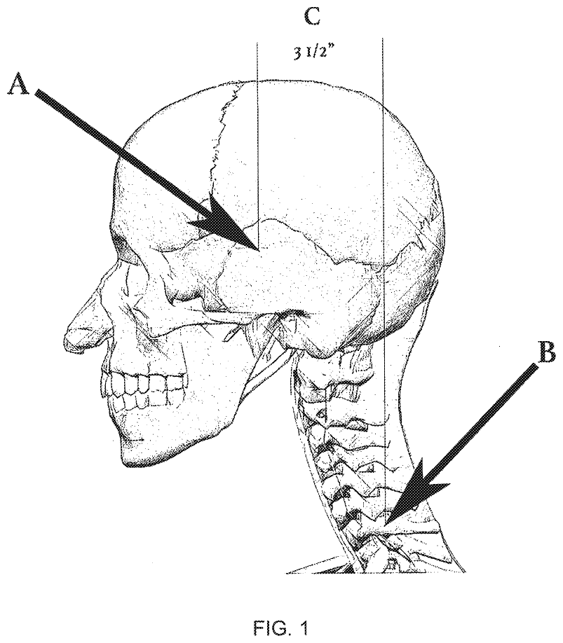

### DETAILED DESCRIPTION

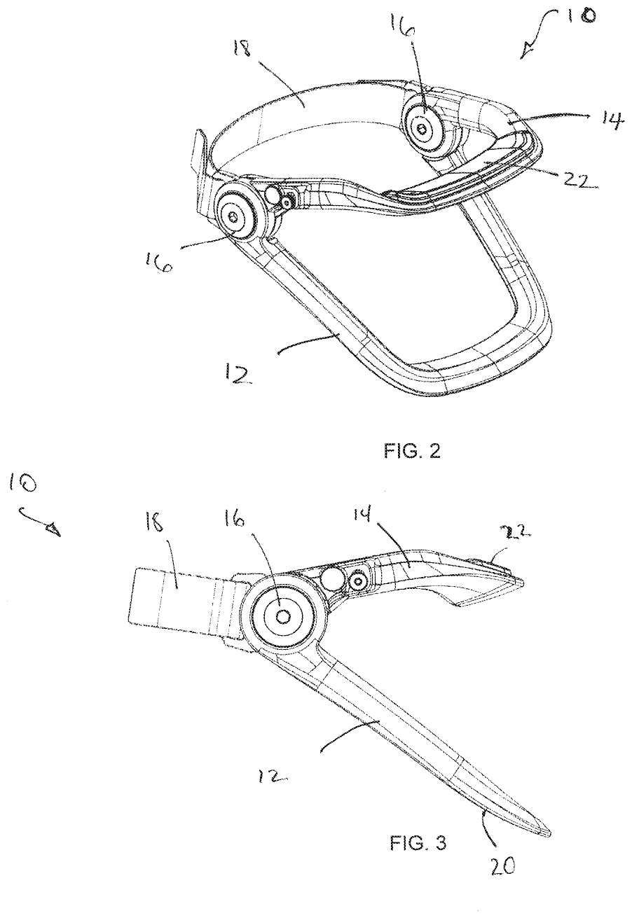

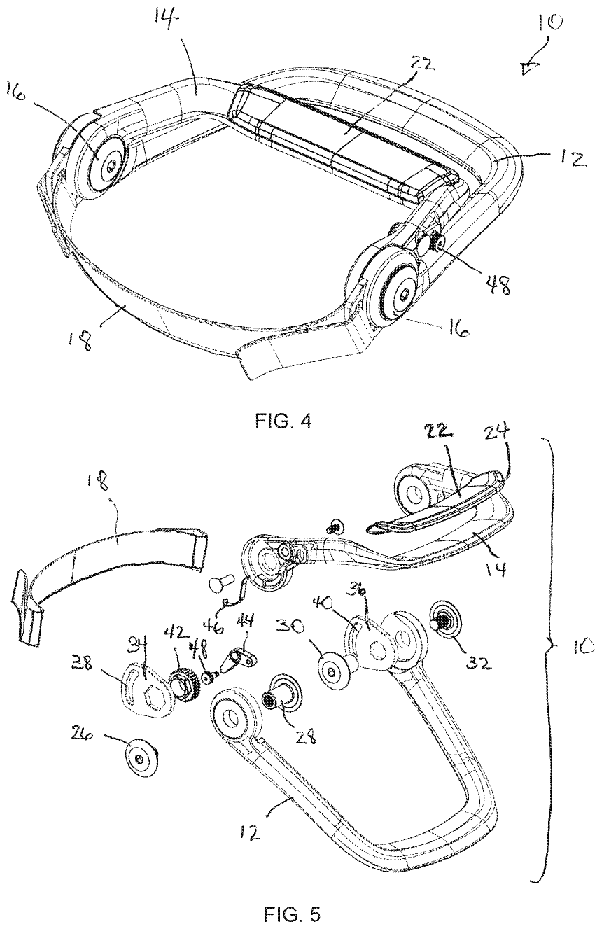

FIGS. 2-6 show one embodiment of a head support according to this invention. The head support 10 has a collapsed configuration for storage and one or more expanded configurations for use. A chest member 12 connects to a chin member 14 at a multi-position connection 16. The multi-position connection 16 maintains the angle between chest member 12 and chin member 14 in the expanded position shown in FIGS. 2 and 3. In some embodiments, the multi-position connection 16 may also maintain the angle between chest member 12 and chin member 14 in the collapsed configuration shown in FIG. 4. In some embodiments, there are a plurality of expanded configurations, each with a different angle between the chest member 12 and the chin member 14 in order to expand the device to a position that is comfortable for the user based on the angle of the user's seat, the shape of the user's chin and/or chest, etc.

A strap 18 extends from first and second sides of the chest member 12 and chin member 14. In some embodiments, the length of strap 18 is adjustable. In some embodiments, strap 18 may be detached from one or both sides of chest member 12 and chin member 14. In this exemplary embodiment, strap 30 is 10-12 inches long and 2-3 inches wide, although other sizes are possible in other embodiments.

In the embodiment illustrated by FIGS. 2-6, chest member 12 and chin member 14 each has an open U shape. Chest member 12 has a bottom surface 20 adapted to engage the user's chest. Chin member 14 has an upper surface 22 adapted to engage the user's chin. In some embodiments, upper surface 22 is on a removable chin support member 24. Chin support member 24 may be formed from a cushioning material, such as foam, polyester, gel, latex, or a combination thereof, and it may be replaced from time to time for hygienic or other reasons.

In some embodiments, multi-position connection 16 has pivot members 26, 28, 30 and 32 permitting pivoting movement between the chest member 12 and the chin member 14. In some embodiments, multi-position connection 16 also includes strap connection elements 34 and 36. The strap 18 is received within slots 38 and 40 in the strap connection elements. In some embodiments, the strap connection elements 34 and 36 may enable the strap to pivot to change the angle of the strap with respect to chest member 12 and/or chin member 14.

In some embodiments, multi-position connection 16 includes a ratchet and ratchet release to maintain and change the angle between the chest member and the chin member. The ratchet includes a toothed gear 42, a pawl 44, a spring 46, and a release actuator 48. Movement of release actuator 48 moves pawl 44 against spring 46 to disengage pawl 44 from toothed gear 42, thereby permitting the angle between chest member 12 and chin member 14 to be changed to and from a collapsed configuration (shown in FIG. 4) and among a plurality of expanded configurations (one of which is shown in FIGS. 2-3).

In an exemplary embodiment, the distance between the bottom of chest member 12 and the top of the chin member of head support 10 is 1.5 inches in its collapsed configuration. The angle between the chest member 12 and the chin member 14 may be increased to an expanded configuration in which the distance between the bottom of chest member 12 and the top of the chin member of the head support 10 is 5 inches. The head support may be adjusted to a plurality of other heights (corresponding to different angles between the chest member 12 and the chin member 14) by adjusting the multi-position connection 16.

The components of the head support of this invention may be made of a variety of materials, such as plastics, aluminum, composites (fiberglass/carbon fiber). A light or luminescent material may be added to help the user find the head support in a dark car or airplane cabin.

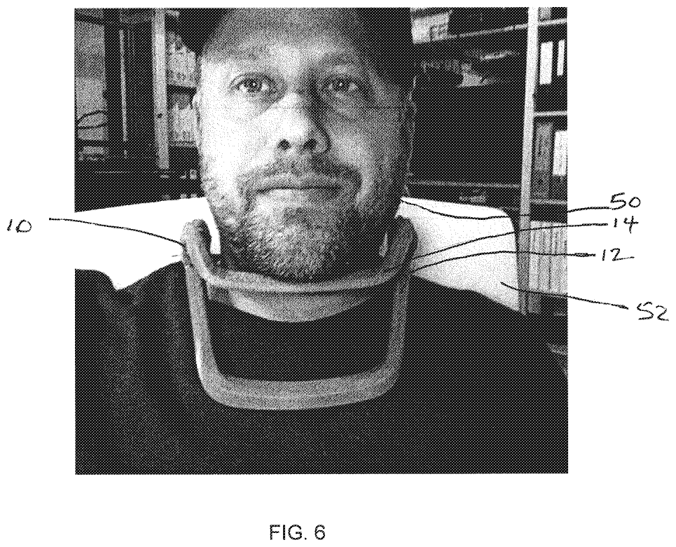

FIG. 6 shows head support 10 supporting the head 50 of a user seated, e.g., in an airline seat. Head support 10 has been expanded from its collapsed configuration to an expanded configuration that is appropriate for the user's anatomy and the user's position with respect to the seat 52 the user is sitting in. After expanding head support 10 to the expanded configuration, the multi-position connection maintains the expanded angle between the chest member 12 and the chin member 14 in the position selected by the user to be the most comfortable for supporting the user's head 50 while sleeping in an upright position. The user's chin rests on the upper surface of chin member 14, and the chest member 12 rests on the user's chest. After use, the ratchet of the multi-position connection may be released so that the head support 10 may be move to its collapsed configuration for storage.

What is claimed is:

1. A head support comprising:
a chin member comprising a chin engagement surface shaped to engage a user's chin;
a chest member comprising a chest engagement surface shaped to engage the user's chest;
a multi-position connection between the chest member and the chin member adapted to maintain an angle between the chest member and the chin member in a collapsed angle of a collapsed configuration and in a plurality of expanded angles greater than the collapsed angle, each expanded angle corresponding to an expanded configuration, the multi-position connection comprising an angle adjustment actuator adapted to permit the angle between the chest member and the chin member to be changed or maintained; and
a strap configured to extend from first and second sides of the chest support and around the user's neck or head when the chest member is engaged with the user's chest.

2. The head support of claim 1 wherein the chest member extends in an open U shape from the multi-position connection.

3. The head support of claim 1 wherein the chin member extends in an open U shape from the multi-position connection.

4. The head support of claim 1 wherein the multi-position connection comprises first and second rotatable connections between the chest member and the chin member disposed at the first and second sides of the chest member and the chin member, respectively.

5. The head support of claim 1 wherein at least one end of the strap is detachable from the head support.

6. The head support of claim 1 wherein the strap has an adjustable operational length.

7. The head support of claim 1 wherein the strap is adapted to pivot with respect to the chest member or the chin member.

8. The head support of claim 1 wherein the chin engagement surface is removable.
